(19) United States
(12) Patent Application Publication
(10) Pub. No.: US 2026/0083444 A1
(43) Pub. Date: Mar. 26, 2026

(54) MEDICAL ANCHORING DEVICE, MEDICAL ANCHORING DEVICE OPERATING METHOD THEREOF AND MEDICAL ANCHORING SYSTEM FOR THE SAME

(71) Applicant: Ribcure Co., Ltd., Tainan City (TW)

(72) Inventors: Ying-Chieh Su, Tainan City (TW); Wei-Chih Lu, Tainan City (TW); Yung-Ching Su, Tainan City (TW); Tse-Yu Su, Tainan City (TW)

(21) Appl. No.: 19/335,182

(22) Filed: Sep. 22, 2025

Related U.S. Application Data
(60) Provisional application No. 63/697,869, filed on Sep. 23, 2024.

(51) Int. Cl. A61B 17/00; A61B 17/34

(52) U.S. Cl. CPC A61B 17/00234; A61B 17/3401; A61B 2017/0034; A61B 2017/00367; A61B 2017/00477

(57) ABSTRACT

A medical anchoring device, comprising: a first wire portion having a first end and a second end; a second wire portion having a third end and a fourth end; a first puncturing portion connecting to the first end of the first wire portion and configured to puncture through a first target position of a cavity wall of a target cavity of a human body; a second puncturing portion connecting to the third end of the second wire portion and configured to puncture through a second target position of the cavity wall of the target cavity, the second target position is different to the first target position; and a connecting portion connecting to the second end of the first wire portion and the fourth end of the second wire, the connecting portion is configured to connect a target object inside the target cavity.

### FIELD OF THE INVENTION

The present disclosure relates to a medical anchoring device for surgery and a medical anchoring device operating method for the same. Particularly, to a medical anchoring device for minimally invasive surgery and/or gasless surgery and the medical anchoring device operating method for the same.

### DESCRIPTION OF THE PRIOR ART

Videoscopic technique was developed in 1980s. Later on, according to the progression of the camera technique and computer technique, the devices were minimized progressively. And the instruments for MIS (minimally invasive surgery) had an outbreak after 2000s. More and more advanced surgery assisted by videoscopy can be accomplished by MIS.

When practicing a MIS, surgeons need to create a space in the body cavity prior to practicing procedures. With a cavitation, surgeons can get a view and let the instruments to have space to operate. Traditionally, the cavity inside body is created by CO2 inflation. The surgeons usually need to set up multiple air seal ports to adapt multiple instruments and scope to perform a surgery.

Later on, more and more surgeons pursue less or even no incisions on belly or chest when they perform MIS. But, how to keep body cavity air seal after an incision is made is always a problem. May LESS (laparo-endoscopic single site) ports were developed in last decade. (All of them have the ability to constraint air leak. But each of them has corresponding defects due to the designed structure. And, none of them have the ability to tent the body cavity without CO2 inflation. None of them could function as a steady adapting point for the surgical instruments.

Furthermore, when using the LESS ports, because all the instruments and videoscope are packing in the same hole, surgeons need to tolerate the instrument collision, low degree of freedom and view shelter.

Of course CO2 inflation technique is a well proved, safe technique to perform MIS. But, long time, high pressure CO2 inflation relates to remarkable elevation of Blood CO2 level. And some literatures proved its risk of patient poor outcome. Furthermore, when using CO2 inflation technique, the suction maneuver during surgery will cause a body cavity collapse. And it's quite troublesome for the surgeons.

According to the above, if there is a gasless technique that can tent the body cavity well, some big issues can be solved. In consideration of this unmet need, some medical devices were developed. For example, some of the inventions focused on how to use an umbrella like structure that introduced outside in through a working port and open in the cavity to lift the abdominal wall, in which the design thinking thereof is to open a lifting device as strong and as wide as possible to perform as a lifting surface. There is one application US20080234551 A1 mentioned a quite similar idea to use hook and wires that applied on the opened surgical incision to do the work of lifting. But this solution is not so perfect, because the lifting position depends on the incision position, which will make the effect of lifting much restricted.

The present invention is meant to solve the problem with a possible gasless surgical system, which may comprises a multi-task adapter with adjustable anchoring. It should be understood that with such an anchoring device, surgeons can acquire a controllable, adjustable, steady connecting point on the cavity wall. It's not only benefit to tent the cavity. It also provides another docking point aside the LESS port. Even more, surgeons might not need a LESS port and have the probability to do a NOTES (nature orifice transluminal endoscopic surgery). The connecting points on the wall might adapt.

FIG. 1 is a section view of abdominal cavity. The whole abdominal cavity is filled with internal organs, including liver 701, left kidney 704, right kidney 702, spleen 705 and stomach 706 inside belly 700. FIG. 1 also shows the position of the spine 703. As illustrated in FIG. 1, there is no space between belly and internal organs, so there is no space for the surgeon to get view or operate the surgical instruments.

FIG. 2 illustrates a distended abdominal cavity (intraperitoneal space). As illustrated in FIG. 2, the internal organs lies in the bottom of the intraperitoneal space 712 due to the gravity. The space between the internal organs and the distended belly 711 facilitates a surgeon for operating a surgery.

FIG. 3 illustrates a traditional way to acquire a distended abdominal cavity by using air sealing trocars 720, 730, 740 and inflated CO2. As illustrated in FIG. 3, CO2 is transmitted into the abdominal cavity through the tube of the air sealing trocars (such as the tube 721 of the air sealing trocar 720), so that the high pressure CO2 distended the abdominal cavity and pushed the abdominal wall (belly) upward to obtain the CO2 inflated space 750, and thereby the surgery can go on. However, the high pressure CO2 will dissolve into blood and cause acidosis. Besides, it is hard to maintain the distended abdominal cavity under this air sealing setting, since the suction work may cause collapse of the distended abdominal cavity.

### SUMMARY OF THE INVENTION

The present invention provides a medical anchoring device, comprising: a first wire portion having a first end and a second end; a second wire portion having a third end and a fourth end; a first puncturing portion connecting to the first end of the first wire portion and configured to puncture through a first target position of a cavity wall of a target cavity of a human body; a second puncturing portion connecting to the third end of the second wire portion and configured to puncture through a second target position of the cavity wall of the target cavity, wherein the second target position is different to the first target position; and a connecting portion connecting to the second end of the first wire portion and the fourth end of the second wire, the connecting portion is configured to connect a target object inside the target cavity.

In a preferred embodiment of the present invention, the connecting portion is a bulldog clamp or a non-traumatic clip or a non-traumatic clamp.

In a preferred embodiment of the present invention, the medical anchoring device further comprising: a third wire portion having a fifth end and a sixth end; and a third puncturing portion connecting to the fifth end of the third wire portion and configured to puncture through a third target position of the cavity wall of the target cavity, wherein the third target position is different to the first target position and the second target position; wherein the sixth end of the third wire portion is connected to the connecting portion.

In a preferred embodiment of the present invention, the medical anchoring device further comprising: a fourth wire portion having a seventh end and an eighth end; and a fourth puncturing portion connecting to the seventh end of the fourth wire portion and configured to puncture through a fourth target position of the cavity wall of the target cavity, wherein the fourth target position is different to the first target position, the second target position and the third target position; wherein the eighth end of the fourth wire portion is connected to the connecting portion.

In a preferred embodiment of the present invention, the medical anchoring device further comprising: a first operating device configured to connect the first end of the first wire portion and/or the first puncturing portion after the first puncturing portion puncturing through the first target position and while the first end is outside the target cavity; and a second operating device configured to connect the third end of the second wire portion and/or the second puncturing portion after the second puncturing portion puncturing through the second target position and while the third end is outside the target cavity.

In a preferred embodiment of the present invention, the connecting portion comprises: a main portion; a first extending portion connected to the main portion; a second extending portion connected to the main portion; and a jaw portion including an upper jaw portion and a lower jaw portion, the upper jaw portion is connected to the first extending portion, and the lower jaw portion is connected to the second extending portion; wherein the upper jaw portion and the lower jaw portion are formed as ring shape.

In a preferred embodiment of the present invention, the upper jaw portion has a first jaw end and a second jaw end, the first jaw end is connected to the first extending portion, and the second jaw end is opposite to the first jaw end; wherein the lower jaw portion has a third jaw end and a fourth jaw end, the third jaw end is connected to the second extending portion, and the fourth jaw end is opposite to the third jaw end; wherein a first thickness of the upper jaw portion is gradually decreased from the first jaw end to the second jaw end; wherein a second thickness of the lower jaw portion is gradually decreased from the third jaw end to the fourth jaw end.

According to an objective of the present invention, the present invention provides a medical anchoring system, comprising: a medical anchoring device as described above; and an endoscopic device configured to place the medical anchoring device inside the target cavity and configured to connect the connecting portion of the medical anchoring device to the target object inside the target cavity.

In a preferred embodiment of the present invention, the endoscopic device comprises: a tube portion; a handle portion connecting the tube portion; a trigger portion connecting the handle portion; and a controlling portion disposed at the end of the tube portion; wherein the medical anchoring device is detachably received at the controlling portion.

In a preferred embodiment of the present invention, the connecting portion has a jaw portion configured to connect the target object; wherein when the trigger portion is triggered, the trigger portion cause the controlling portion to control the jaw portion into an open state.

In a preferred embodiment of the present invention, the endoscopic device is configured to make the first puncturing portion puncturing through the first target position of the cavity wall, and the endoscopic device is configured to make the second puncturing portion puncturing through the second target position of the cavity wall.

In a preferred embodiment of the present invention, the medical anchoring device is a first medical anchoring device, the connecting portion of the first medical anchoring device is a first connecting portion, the target object is a first target object; wherein the medical anchoring system comprise a second medical anchoring device, and the second medical anchoring device comprises: a fifth wire portion having a ninth end and a tenth end; a sixth wire portion having a eleventh end and a twelfth end; a fifth puncturing portion connecting to the ninth end of the fifth wire portion and configured to puncture through a fifth target position of the cavity wall of the target cavity; a sixth puncturing portion connecting to the eleventh end of the sixth wire portion and configured to puncture through a sixth target position of the cavity wall of the target cavity, wherein the sixth target position is different to the fifth target position; and a second connecting portion connecting to the tenth end of the fifth wire portion and the twelfth end of the sixth wire, the second connecting portion is configured to connect the first target object or a second target object inside the target cavity.

In a preferred embodiment of the present invention, the endoscopic device is configured to make the fifth puncturing portion puncturing through the fifth target position of the cavity wall, and the endoscopic device is configured to make the sixth puncturing portion puncturing through the sixth target position of the cavity wall.

According to an objective of the present invention, the present invention provides a method for operating a medical anchoring device, comprising the steps of: placing the medical anchoring device into a target cavity of a human body by using an endoscopic device, wherein the anchoring device comprises a first wire portion, a second wire portion, a first puncturing portion, a second puncturing portion and a connecting portion, the first puncturing portion connects a first end of the wire portion, the second puncturing portion connects a third second end of the wire portion, and the connecting portion connects a second end of the first wire portion and a fourth end of the second wire portion; puncturing, by the first puncturing portion, through a first target position of a cavity wall of the target cavity; puncturing, by the second puncturing portion, through a second target position of the cavity wall of the target cavity, wherein the first target position is different to the second target position; connecting the connecting portion to a target object inside the target cavity; and pulling the first wire portion and/or the second wire portion, so as to move the target object to a desired position or move the target object toward a desired direction.

In a preferred embodiment of the present invention, the anchoring device comprises a third wire portion and a third puncturing portion, the third puncturing portion is connected to a fifth end of the third wire portion, and a sixth end of the third wire portion is connected to the connecting portion; wherein the method further comprises the step of: puncturing, by the third puncturing portion, through a third target position of a cavity wall of the target cavity, wherein the third target position is different to the first target position and the second target position; and pulling the first wire portion and/or the second wire portion and/or the third puncturing portion, so as to move the target object to the desired position or move the target object toward the desired direction.

In a preferred embodiment of the present invention, the anchoring device comprises a fourth wire portion and a fourth puncturing portion, the fourth puncturing portion is connected to a seventh end of the fourth wire portion, and an eighth end of the fourth wire portion is connected to the connecting portion; wherein the method further comprises the step of: puncturing, by the fourth puncturing portion, through a fourth target position of a cavity wall of the target cavity, wherein the fourth target position is different to the first target position, the second target position and the third target position; and pulling the first wire portion and/or the second wire portion and/or the third puncturing portion and/or the fourth puncturing portion, so as to move the target object to the desired position or move the target object toward the desired direction.

### BRIEF DESCRIPTION OF THE DRAWINGS

FIG. 1 is a section view of abdominal cavity.

FIG. 2 is a section view of a distended abdominal cavity (intraperitoneal space).

FIG. 3 is a section view of a distended abdominal cavity acquired by using traditional air sealing trocars and inflated CO2.

FIG. 4 is a schematic diagram of a medical anchoring device in accordance with an embodiment of the present invention.

FIG. 5 is a schematic diagram of a medical anchoring device in accordance with an embodiment of the present invention.

FIG. 6 is a flow diagram of a method for operating a medical anchoring device in accordance with an embodiment of the present invention.

FIG. 7 is a schematic diagram illustrating the operation of a medical anchoring device in accordance with an embodiment of the present invention.

FIG. 8 is a schematic diagram of a medical anchoring system in accordance with an embodiment of the present invention.

FIG. 9A is a schematic diagram of a connecting portion in accordance with an embodiment of the present invention.

FIG. 9B is a schematic diagram of a connecting portion in accordance with an embodiment of the present invention.

FIG. 9C is a schematic diagram of a connecting portion in accordance with an embodiment of the present invention.

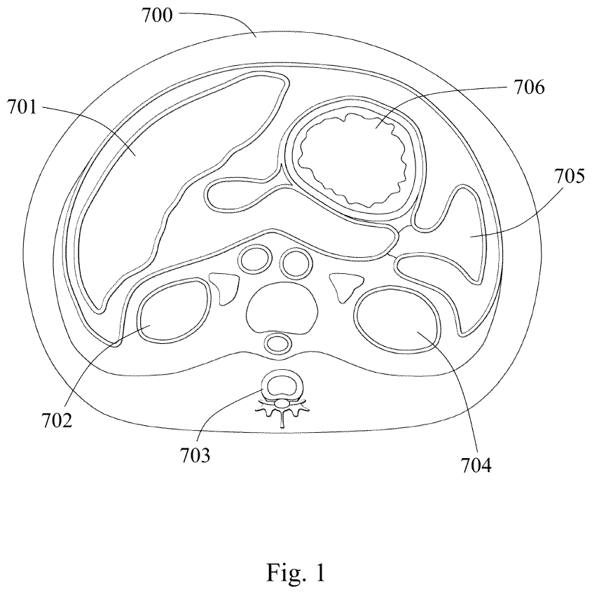

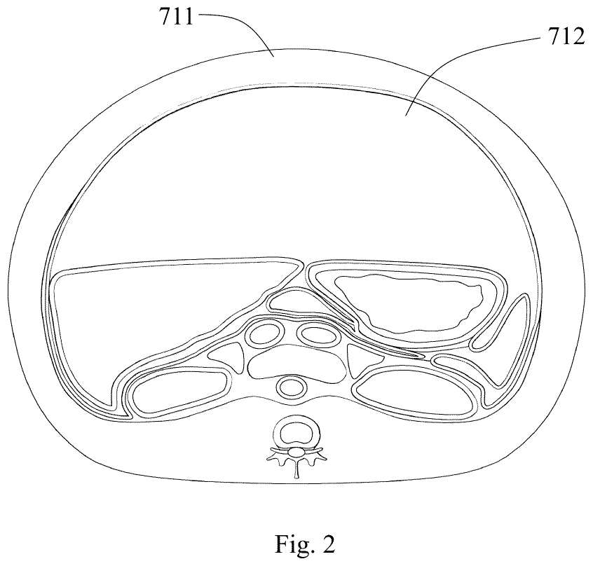

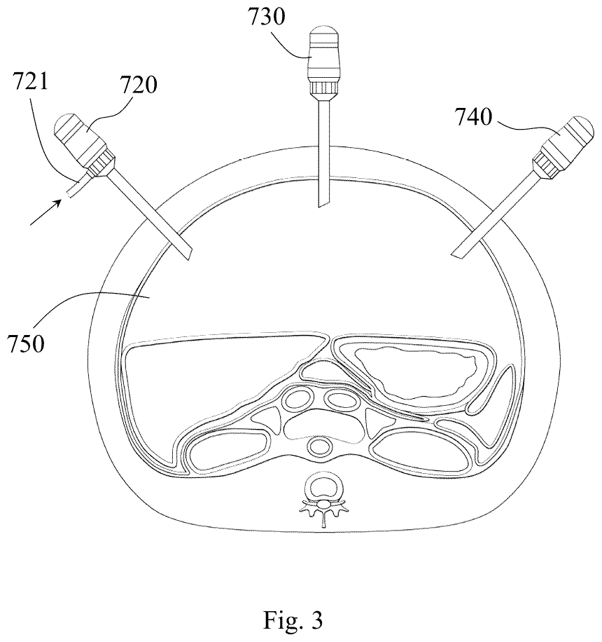

### DETAILED DESCRIPTION OF THE EMBODIMENTS

The present invention will be described more fully hereinafter with reference to the accompanying drawings, and some embodiments are illustrated by way of example. However, the claimed subject matter may be embodied in various forms, and thus the construction of the claimed subject matter is not limited to any embodiments disclosed in this specification; the embodiments described for illustrative purposes are exemplary only. Similarly, the present invention is intended to provide a reasonably broad scope for the claimed subject matter that is covered herein.

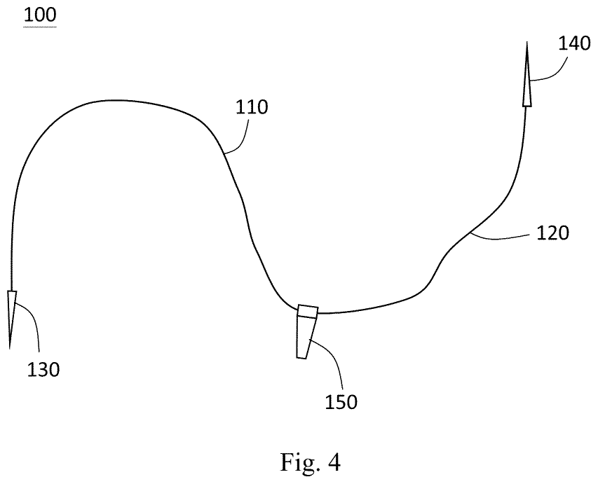

Please refer to FIG. 4, which is a schematic diagram of a medical anchoring device in accordance with an embodiment of the present invention. As the embodiment in FIG. 4 shows, the medical anchoring device 100 (the medical anchoring device 100 may refer as first medical anchoring device) comprises the first wire portion 110, the second wire portion 120, the first puncturing portion 130, the second puncturing portion 140 and the connecting portion 150 (the connecting portion 150 may refer as first connecting portion). The first puncturing portion 130 is connected to one end (which may be referred to as a first end) of the first wire portion 110, and the connecting portion 150 is connected to the other end (which may be referred to as a second end) of the first wire portion 110. The second puncturing portion 140 is connected to one end (may be referred to as a third end) of the second wire portion 120, and the connecting portion 150 is connected to the other end (which may be referred to as a fourth end) of the second wire portion 120. It should be understood that the first wire portion 110 and the second wire portion 120 may be any kind of wire or any kind of string according to the needs, and is not limited thereto.

The first puncturing portion 130 is configured to puncture through (i.e. penetrate) a first target position of a cavity wall of a target cavity of a human body (the target cavity may be the abdominal cavity of the human body, but is not limited thereto), the second puncturing portion 140 is configured to puncture through (i.e. penetrate) a second target position of the cavity wall of the target cavity, and the connecting portion 150 is configured to connect a target object (may refer as first target object) inside the target cavity.

Preferably, the first puncturing portion 130 and the second puncturing portion 140 are needles, but are not limited thereto. Preferably, the first target position is different from the second target position. Preferably, the connecting portion 150 is detachably connected to the target object, so that the connecting portion 150 can be removed from the target object and the cavity after the surgery. The target object may be an organ or a tissue inside a target cavity of a human or an animal. For example, the target object may be an intestine of a human body, but is not limit thereto. Preferably, the connecting portion 150 may be a clamping device configured to clamp the target object inside the target cavity (in which the target object may be an organ or a tissue or a medical apparatus inside the target cavity. For example, the target cavity may be an abdominal cavity, and the target object may be an organ inside the abdominal cavity). For example, the connecting portion 150 may be a bulldog clamp or a non-traumatic clip or a non-traumatic clamp or any other medical clamp, but is not limited thereto. Preferably, the connecting portion 150 has a jaw portion for connecting (such as clamping) the target object. It should be understood that the connecting portion can be made by metal or plastic according to the needs, but is not limited thereto. It should be understood that the number of wire portions and the number of puncturing portions can be provided depending on the needs; for example, there can be two or more wire portions and puncturing portions, and each of the wire portion may puncture through (i.e. penetrate) a different target position of the cavity wall of the target cavity by using a corresponding puncturing portion. Preferably, the number of the wire portions is the same to the number of the puncturing portion, and each wire portion is connecting to a corresponding puncturing portion. It should be understood that the number of connecting portion can be provided depending on the needs; for example, there can be one or more connecting portions. In one embodiment, the medical anchoring device has two connecting portions, in which both the connecting portions are preferably connected to all of the wire portions of the medical anchoring device.

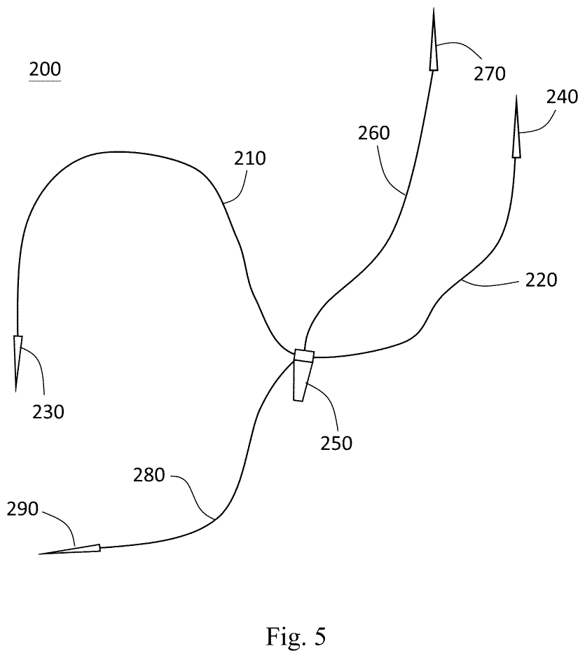

Please refer to FIG. 5, which is a schematic diagram of a medical anchoring device in accordance with an embodiment of the present invention. As the embodiment in FIG. 5 shows, the medical anchoring device 200 comprises first wire portion 210, second wire portion 220, third wire portion 260, fourth wire portion 280, first puncturing portion 230, second puncturing portion 240, third puncturing portion 270, fourth puncturing portion 290 and connecting portion 250.

In one embodiment, the medical anchoring device may further comprises a first operating device and a second operating device. The first operating device is configured to connect the first end of the first wire portion and/or the first puncturing portion after the first puncturing portion puncturing through the first target position and while the first end is outside the target cavity. The second operating device is configured to connect the third end of the first wire portion and/or the second puncturing portion after the second puncturing portion puncturing through the second target position and while the third end is outside the target cavity. As such, the user (such as a surgeon or a robotic surgery system) can hold the first operating device, and control (such as pull) the first wire through the first operating device. Similarly, the surgeon can hold the second operating device, and control (such as pull) the second wire through the second operating device. The first operating device and the second operating device may be a grip, but is not limited thereto.

According to FIG. 5, the first puncturing portion 230 is connected to one end (which may be referred to as a first end) of the first wire portion 210, and the connecting portion 250 is connected to the other end (which may be referred to as a second end) of the first wire portion 210. The second puncturing portion 240 is connected to one end (may be referred to as a third end) of the second wire portion 220, and the connecting portion 250 is connected to the other end (which may be referred to as a fourth end) of the second wire portion 220. The third puncturing portion 270 is connected to one end (may be referred to as a fifth end) of the third wire portion 260, and the connecting portion 250 is connected to the other end (which may be referred to as a sixth end) of the third wire portion 260. The fourth puncturing portion 290 is connected to one end (may be referred to as a seventh end) of the fourth wire portion 280, and the connecting portion 250 is connected to the other end (which may be referred to as an eighth end) of the fourth wire portion 280. It should be understood that the first wire portion 210, the second wire portion 220, the third wire portion 260 and the fourth wire portion 280 may be any kind of wire or any kind of string according to the needs, and is not limited thereto.

The first puncturing portion 230 is configured to puncture through (i.e. penetrate through) a first target position of a cavity wall of a target cavity of a human body (the target cavity may be the abdominal cavity of the human body, but is not limited thereto), the second puncturing portion 240 is configured to puncture through (i.e. penetrate through) a second target position of the cavity wall of the target cavity, the third puncturing portion 270 is configured to puncture through (i.e. penetrate through) a third target position of the cavity wall of the target cavity, the fourth puncturing portion 290 is configured to puncture through (i.e. penetrate through) a fourth target position of the cavity wall of the target cavity, and the connecting portion 250 is configured to connect a target object inside the target cavity.

Preferably, the first puncturing portion 230, the second puncturing portion 240, the third puncturing portion 270 and the fourth puncturing portion 290 are needles, but are not limited thereto. Preferably, the first target position, the second target position, the third target position and the fourth target position are different positions on the cavity wall of the target cavity (that is to say, the first target position is different from the second target position, the third target position and the fourth target position; the second target position is different from the first target position, the third target position and the fourth target position; and the third target position is different from the first target position, the second target position and the fourth target position).

Preferably, the connecting portion 250 is detachably connected to the target object, so that the connecting portion 250 can be removed from the target object and the cavity after the surgery. Preferably, the connecting portion 250 may be a clamping device configured to clamp the target object inside the target cavity (in which the target object may be an organ or a tissue or a medical apparatus inside the target cavity. For example, the target cavity may be an abdominal cavity, and the target object may be an organ inside the abdominal cavity). For example, the connecting portion 250 may be a bulldog clamp or a non-traumatic clip or a non-traumatic clamp or any other medical clamp, but is not limited thereto. It should be understood that the number of wire portions and the number of puncturing portions can be provided depending on the needs; for example, there can be two or more than three wire portions and puncturing portions.

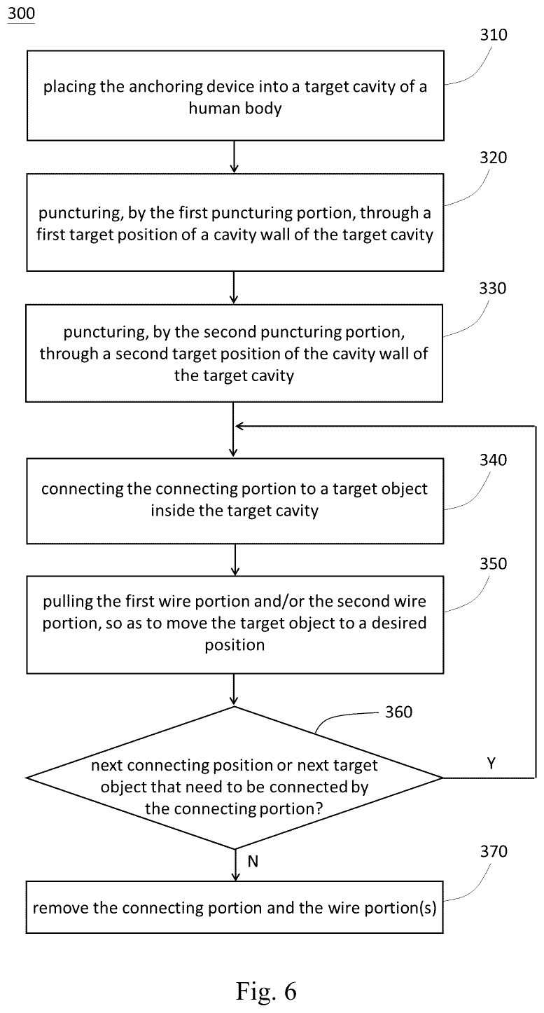

Referring to FIG. 6, a flow diagram of an embodiment of the method for operating a medical anchoring device of the present invention is provided. As shown in FIG. 5, the method 300 for operating a medical anchoring device starts from the step 310, placing the anchoring device into a target cavity (such as an abdominal cavity, but is not limited thereto) of a human body. Preferably, the medical anchoring device may be placed into the target cavity by using an endoscopic device, but is not limited thereto. The endoscopic device may be a laparoscopic clip applier or a bulldog clamp applier or a non-traumatic clip applier or a non-traumatic clamp applier, but is not limited thereto. It should be understood that the medical anchoring device may be placed into the target cavity by using other device or by using other way according to the needs.

Next, the step 320 is performed, puncturing (penetrating), by the first puncturing portion, through a first target position of a cavity wall of the target cavity. Preferably, the first puncturing portion is punctured from inside of the target cavity to the outside of the target cavity. Preferably, the first puncturing portion may be punctured through the first target position of the cavity wall by using the endoscopic device (for example, the first puncturing portion may be detachable connected to the endoscopic device, and the user such as a surgeon may control the endoscopic device to push the first puncturing portion, so as to make the first puncturing portion puncturing through the first target position), but is not limited thereto. It should be understood that the first puncturing portion may be punctured through the first target position of the cavity wall by using other device or by using other way according to the needs. Next, the step 330 is performed, puncturing (penetrating), by the second puncturing portion, through a second target position of the cavity wall of the target cavity, wherein the first target position is different to the second target position. Preferably, the second puncturing portion is punctured from inside of the target cavity to the outside of the target cavity. Preferably, the second puncturing portion may be punctured through the second target position of the cavity wall by using the endoscopic device (for example, the second puncturing portion may be detachable connected to the endoscopic device, and the user such as a surgeon may control the endoscopic device to push the second puncturing portion, so as to make the second puncturing portion puncturing through the second target position), but is not limited thereto. It should be understood that the second puncturing portion may be punctured through the second target position of the cavity wall by using other device or by using other way according to the needs.

Next, the step 340 is performed, connecting the connecting portion to a target object inside the target cavity. Preferably, the connecting portion may be connected to the target object by using the endoscopic device, but is not limited thereto. It should be understood that the connecting portion may be connected to the target object by using other device or by using other way according to the needs. Next, the step 350 is performed, pulling the first wire portion and/or the second wire portion (such as pulling the first end of the first wire portion and/or the third end of the second wire portion), so as to move the target object to a desired position or move the target object toward a desired direction. Next, the step 360 is performed, the surgeon may decide whether it needs to change the target connecting position of the connecting portion, or whether there is next target object that need to be connected and controlled by the connecting portion (step 360). If the answer of the step 360 is yes, then step 340 is performed (and steps 350, 360 are performed again after that), the surgeon may use the connecting portion to connect the next target connecting position of the present target object or to connect the next target object inside the target cavity. Otherwise, if there is no next target connecting position that need to be connected and controlled by the connecting portion, and there is no next target object that need to be connected and controlled by the connecting portion, then the step 370 is performed, in which the use of the connecting portion in the surgery is finished, so the surgeon may remove the connecting portion and the wire portion(s) from the target cavity. It should be understood that the step 340 to the step 360 is a loop, which means the step 340, step 350 and step 360 may be performed repeatedly until the answer of the step 360 is no and the step 370 is performed.

In one embodiment, while the use of the connecting portion is finished and the step 370 is performed, the surgeon may cut off all the wire portion from the outside of the target cavity, and then remove the connecting portion out of the target cavity by using any kind of removal device (such as an endoscopic device). Preferably, the puncturing portions do not enter the target cavity again during the removal procedure of the connecting portion.

In one embodiment, the anchoring device may comprises a third wire portion and a third puncturing portion, in which the third puncturing portion is connected to a fifth end of the third wire portion, and a sixth end of the third wire portion is connected to the connecting portion. The method 300 may further comprises the step of: puncturing (penetrating), by the third puncturing portion, through a third target position of a cavity wall of the target cavity. Wherein the third target position is different to the first target position and the second target position. Also, the method 300 may further comprises the step of: pulling the first wire portion and/or the second wire portion and/or the third puncturing portion, so as to move the target object to the desired position or move the target object toward the desired direction.

In one embodiment, the anchoring device may further comprises a fourth wire portion and a fourth puncturing portion, in which the fourth puncturing portion is connected to a seventh end of the fourth wire portion, and an eighth end of the fourth wire portion is connected to the connecting portion. The method 300 may further comprises the step of: puncturing (penetrating), by the fourth puncturing portion, through a fourth target position of a cavity wall of the target cavity. Wherein the fourth target position is different to the first target position, the second target position and the third target position. Also, the method 300 may further comprises the step of: pulling the first wire portion and/or the second wire portion and/or the third puncturing portion and/or the fourth puncturing portion, so as to move the target object to the desired position or move the target object toward the desired direction.

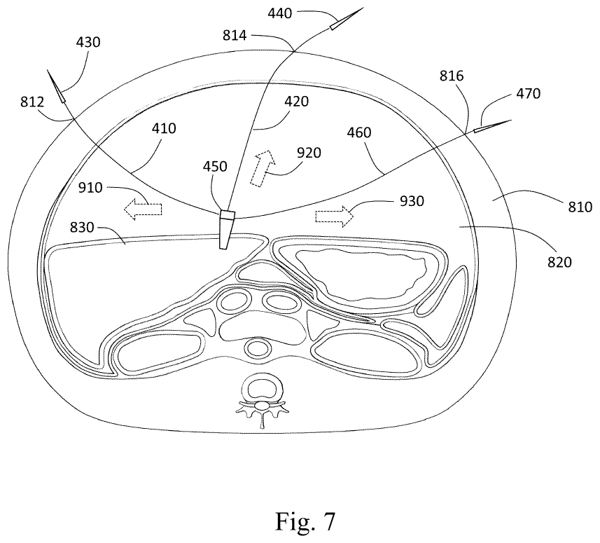

Please refer to FIG. 7, which is a schematic diagram illustrating the operation of a medical anchoring device in accordance with an embodiment of the present invention. As the embodiment in FIG. 7 shows, the connecting portion 450 of the medical anchoring device is placed into the abdominal cavity 820 of a human body, and the connecting portion 450 clamped the target organ 830 (the target organ 830 is the target object in this case) inside the abdominal cavity 820. The first puncturing portion 430 with the first wire portion 410 punctured through the first target position 812 of the cavity wall 810, the second puncturing portion 440 with the second wire portion 420 punctured through the second target position 814 of the cavity wall 810, the third puncturing portion 470 with the third wire portion 460 punctured through the third target position 816 of the cavity wall 810.

Under the arrangement of the medical anchoring device as shown in FIG. 7, the surgeon may move the target organ 830 with the connecting portion 450 to a desired position or toward a desired direction by pulling the first wire portion 410 and/or the second wire portion 420 and/or the third wire portion 460 and/or the first puncturing portion 430 and/or the second puncturing portion 440 and/or the third puncturing portion 470.

For example, the surgeon may move the target organ 830 with the connecting portion 450 toward direction 910 by pulling the first wire portion 410 and/or the first puncturing portion 430. Or, the surgeon may move the target organ 830 with the connecting portion 450 toward direction 920 by pulling the second wire portion 420 and/or the second puncturing portion 440. Or, the surgeon may move the target organ 830 with the connecting portion 450 toward direction 930 by pulling the third wire portion 460 and/or the third puncturing portion 470.

In one embodiment, each of the puncturing portion may be replaced with an operating device (such as another clamping device or a grip or a grabbing device, but is not limited thereto) after the puncturing portion and the wire portion are punctured through the target position of the cavity wall. Selectively, each of the wire portion may be connected to an operating device As such, the surgeon can easily control the wire portion by operating the operating device.

In one embodiment, after the connecting portion is connected to the target object or target organ, and each of the puncture portion that connected with the corresponding wire portion has punctured through the corresponding position of the cavity wall, the user may connect each of the wire portion with an operation portion, so that the operation portion can be used to control the length of the wire portion inside the cavity. For example, the operation portion may be a clamp device that clamp the wire portion and placed on the skin of the cavity, or the operation portion may be an electrical device that used to automatically or non-automatically control the length of the wire portion inside the cavity by pulling or releasing the wire portion, or the operation portion may be a wire reel that used to control the length of the wire portion inside the cavity.

In one embodiment, after the use of the medical anchoring device is finished, the surgeon may cut off all the wire portion (i.e. the first wire portion 410, the second wire portion 420 and the third wire portion 460 in the case of FIG. 7), and then remove the connecting portion out of the target cavity by using any kind of removal device (such as an endoscopic device, but is not limited thereto). Preferably, the puncturing portions do not enter the target cavity again during the removal procedure of the connecting portion.

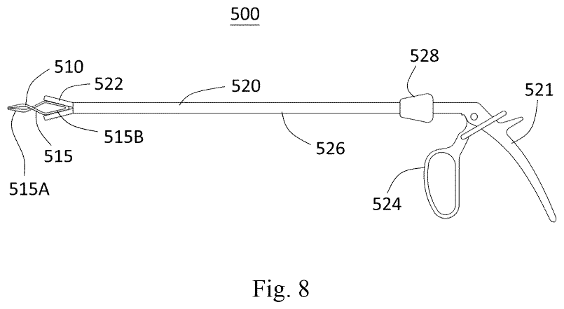

Please refer to FIG. 8, which is a schematic diagram of a medical anchoring system in accordance with an embodiment of the present invention. As the embodiment in FIG. 8 shows, the medical anchoring system 500 comprises a medical anchoring device 510 (the structure and mechanism of the medical anchoring device 510 can refer as above) and an endoscopic device 520. The endoscopic device 520 comprises a handle portion 521, a trigger portion 524, a tube portion 526 and a controlling portion 522. The controlling portion 522 connects the tube portion 526 and is disposed at the end of the tube portion 526. The handle portion 521 connects the tube portion 526, the trigger portion 524 connects the handle portion 521 and is movable or rotatable with respect to the handle portion 521.

As shown in FIG. 8, the medical anchoring device 510 is detachably connected or received at the controlling portion 522 of the endoscopic device 520. The surgeon may press the trigger portion 524 of the endoscopic device 520 to control the connecting portion 515 of the medical anchoring device 510 into an open state (in the open state, the jaw portion of the connecting portion 515 is open). Then, the surgeon may control the connecting portion 515 to contact a target object by operating and/or moving the endoscopic device 520, and the surgeon may release the trigger portion 524 of the endoscopic device 520 to control the connecting portion 515 of the medical anchoring device 510 into a close state (in the close state, the jaw portion of the connecting portion 515 is close), so as to connect the connecting portion 515 with the target object. Also, the surgeon may use the endoscopic device 520 to remove the connecting portion 515 from the target object. Specifically, the surgeon may operate the endoscopic device 520 to receive the connecting portion 515 in the controlling portion 522. Then, the surgeon may press the trigger portion 524 to control the connecting portion 515 of the medical anchoring device 510 into a first state, so that the connecting portion 515 can be easily and safely removed from the target object. After that, the surgeon may remove the connecting portion 515 by the controlling portion 522 of the endoscopic device 520 out of the target cavity.

Preferably, the connecting portion 515 of the medical anchoring device 510 comprises a jaw portion 515A and a pressing portion 515B, the pressing portion 515B connects the jaw portion 515A. Wherein when the surgeon press the trigger portion 524, the controlling portion 522 exerts a pressing force to the pressing portion 515B, so that the jaw portion 515A is opened based on the pressing force and the connecting portion 515 come into the first state (may be refer as opened state or triggered state). Also, when the surgeon release the trigger portion 524, the controlling portion 522 stop exerting the pressing force to the pressing portion 515B, so that the jaw portion 515A is closed and the connecting portion 515 come into the second state (may be refer as closed state or released state). Preferably the jaw portion of the connecting portion formed as a non-traumatic clip or a non-traumatic clamp that does not hurt or damage the target object.

Preferably, the endoscopic device 520 may have a rotational portion 528. The rotational portion 528 is arranged between the handle portion 521 and the tube portion 526, and the rotational portion 528 is rotatable with respect to the handle portion 521 and the tube portion 526, in which the controlling portion 522 is rotate with the rotational portion 528, so that surgeon can operate (such as rotate) the rotational portion 528 to rotate the connecting portion 515 into a desired angle.

In one embodiment, each of the puncturing portion of the medical anchoring device 510 may be detachably connected on the endoscopic device 520. The surgeon may control the endoscopic device 520 to make each of the puncturing portion puncturing through the cavity wall at a corresponding target position. That is to say, according to the needs, the endoscopic device may be configured to make the first puncturing portion puncturing through the first target position of the cavity wall, and the endoscopic device may be configured to make the second puncturing portion puncturing through the second target position of the cavity wall. For example, the first puncturing portion may be detachable connected to the endoscopic device, and the user such as a surgeon may control the endoscopic device to push the first puncturing portion, so as to make the first puncturing portion puncturing through the first target position. Similarly, the second puncturing portion may be detachable connected to the endoscopic device, and the user such as a surgeon may control the endoscopic device to push the second puncturing portion, so as to make the second puncturing portion puncturing through the second target position. It should be understood that the third puncturing portion (if there is one), the fourth puncturing portion (if there is one) and the other puncturing portion (if there is one) can respectively puncture through a corresponding target position by using the same way as to the first puncturing portion and the second puncturing portion.

Preferably, at least a portion of the wire portion of the medical anchoring device 510 is received in the tube portion 526 of the endoscopic device 520 during the puncturing procedure of the puncturing portion. In another embodiment, each of the wire portion and/or at least a portion of each puncturing portion of the medical anchoring device 510 is received in the tube portion 526. The surgeon may operate the endoscopic device 520 to push the puncturing portion out of the tube portion 526 and make the puncturing portion puncturing through the cavity wall.

It should be understand that the medical anchoring system may comprise more than one medical anchoring device according to the needs, so that the surgeon can connect each medical anchoring device to different position on the target object, or the surgeon can connect each medical anchoring device to different target objects. For example, the medical anchoring system may comprises a second medical anchoring device. The second medical anchoring device may comprises a fifth wire portion, a sixth wire portion, a fifth puncturing portion, a sixth puncturing portion and a second connecting portion. The fifth wire portion may have a ninth end and a tenth end, and the sixth wire portion may have a eleventh end and a twelfth end. The fifth puncturing portion is connected to the ninth end of the fifth wire portion and is configured to puncture through a fifth target position of the cavity wall of the target cavity. The sixth puncturing portion is connected to the eleventh end of the sixth wire portion and is configured to puncture through a sixth target position of the cavity wall of the target cavity, wherein the sixth target position is different to the fifth target position. The second connecting portion is connected to the tenth end of the fifth wire portion and the twelfth end of the sixth wire, and the second connecting portion is configured to connect the first target object or a second target object inside the target cavity. Preferably, The endoscopic device is configured to make the fifth puncturing portion puncturing through the fifth target position of the cavity wall, and the endoscopic device is configured to make the sixth puncturing portion puncturing through the sixth target position of the cavity wall.

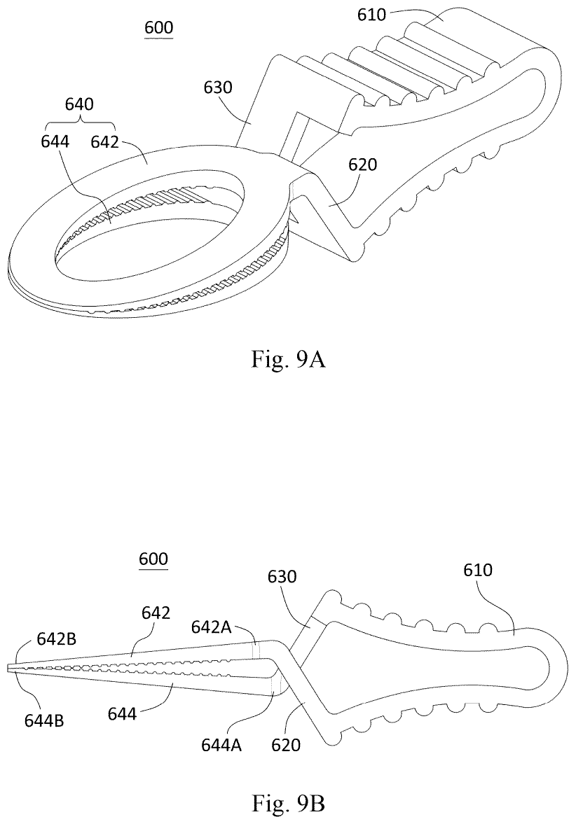

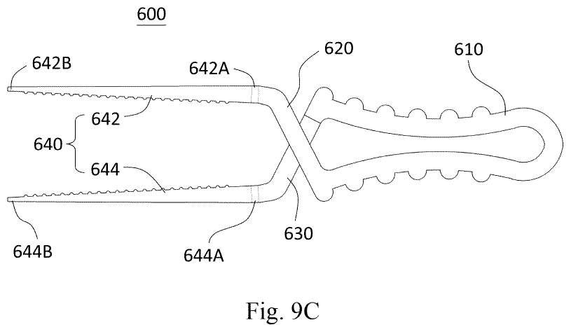

Please refer to FIGS. 9A-9C, which are schematic diagrams of a connecting portion in accordance with an embodiment of the present invention. As the embodiment in FIGS. 9A-9C shows, the connecting portion 600 comprises a main portion 610, a first extending portion 620, a second extending portion 630 and a jaw portion 640, and the jaw portion 640 includes an upper jaw portion 642 and a lower jaw portion 644. The upper jaw portion 642 is connected to the first extending portion 620, and the lower jaw portion 644 is connected to the second extending portion 630. Preferably, the upper jaw portion 642 is formed as ring shape, and the lower jaw portion 644 is also formed as ring shape. It should be understood that the ring shaped jaw portion can reduce damage to the target object while the jaw portion is clamping the target object.

As shown in FIGS. 9B, 9C, the upper jaw portion 642 has a first jaw end 642A and a second jaw end 642B, the first jaw end 642A is connected to the first extending portion 620, and the second jaw end 642B is opposite to the first jaw end 642A. Also, the lower jaw portion 644 has a third jaw end 644A and a fourth jaw end 644B, the third jaw end 644A is connected to the second extending portion 630, and the fourth jaw end 644B is opposite to the third jaw end 644A. Preferably, the thickness (refer as first thickness) of the upper jaw portion 642 is gradually decreased from the first jaw end 642A to the second jaw end 642B, and the thickness (refer as second thickness) of the lower jaw portion 644 is gradually decreased from the third jaw end 644A to the fourth jaw end 644B.

Preferably, the main portion 610 may have a plurality of convex portion (refer as first convex portion) disposed on its outer surface. While the main portion 610 is connected or received at the controlling portion of the endoscopic device, the plurality of convex portion of the main portion 610 contact the controlling portion, so as to increase the friction between the main portion 610 and the controlling portion, and thus ensure the main portion 610 being stably held by the controlling portion.

Preferably, the upper jaw portion 642 may have a plurality of convex portion (refer as second convex portion) disposed on the first contacting surface of the upper jaw portion 642, and the lower jaw portion 644 may have a plurality of convex portion (refer as third convex portion) disposed on the second contacting surface of the lower jaw portion 644. As such, while the jaw portion 640 of the connecting portion 600 is clamping the target object, the plurality of second convex portion of the upper jaw portion 642 and the plurality of third convex portion of the lower jaw portion 644 contact the target object, so as to increase the friction between the jaw portion 640 and the target object, and thus ensure the target object being stably clamped by the jaw portion 640.

Preferably, the above mentioned “puncture through a target position of a cavity wall” means to penetrate through the cavity wall at the target position of a cavity wall, the wording “puncture through” may refer as “penetrate through”. The medical anchoring device, medical anchoring device operating method thereof and medical anchoring system for the same of the present invention has been explained in detail by the above description and drawings. However, it should be understood that various embodiments of the present invention are only used for illustration. Various changes can be made without departing from the spirit and scope of the present invention, and such changes should be covered by the scope of the present invention. Therefore, the embodiments described herein are not intended to limit the present invention, and the true scope and spirit of the present invention are disclosed in the following claims.

What is claimed is:

1. A medical anchoring device, comprising:
a first wire portion having a first end and a second end;
a second wire portion having a third end and a fourth end;
a first puncturing portion connecting to the first end of the first wire portion and configured to puncture through a first target position of a cavity wall of a target cavity of a human body;
a second puncturing portion connecting to the third end of the second wire portion and configured to puncture through a second target position of the cavity wall of the target cavity, wherein the second target position is different to the first target position; and
a connecting portion connecting to the second end of the first wire portion and the fourth end of the second wire, the connecting portion is configured to connect a target object inside the target cavity.

2. The medical anchoring device according to claim 1, wherein the connecting portion is a bulldog clamp or a non-traumatic clip or a non-traumatic clamp.

3. The medical anchoring device according to claim 1, further comprising:
a third wire portion having a fifth end and a sixth end; and
a third puncturing portion connecting to the fifth end of the third wire portion and configured to puncture through a third target position of the cavity wall of the target cavity, wherein the third target position is different to the first target position and the second target position;
wherein the sixth end of the third wire portion is connected to the connecting portion.

4. The medical anchoring device according to claim 3, further comprising:
a fourth wire portion having a seventh end and an eighth end; and
a fourth puncturing portion connecting to the seventh end of the fourth wire portion and configured to puncture through a fourth target position of the cavity wall of the target cavity, wherein the fourth target position is different to the first target position, the second target position and the third target position;
wherein the eighth end of the fourth wire portion is connected to the connecting portion.

5. The medical anchoring device according to claim 1, further comprising:
a first operating device configured to connect the first end of the first wire portion and/or the first puncturing portion after the first puncturing portion puncturing through the first target position and while the first end is outside the target cavity; and
a second operating device configured to connect the third end of the second wire portion and/or the second puncturing portion after the second puncturing portion puncturing through the second target position and while the third end is outside the target cavity.

6. The medical anchoring device according to claim 1, wherein the connecting portion comprises:
a main portion;
a first extending portion connected to the main portion;
a second extending portion connected to the main portion; and
a jaw portion including an upper jaw portion and a lower jaw portion, the upper jaw portion is connected to the first extending portion, and the lower jaw portion is connected to the second extending portion;
wherein the upper jaw portion and the lower jaw portion are formed as ring shape.

7. The medical anchoring device according to claim 1, wherein the upper jaw portion has a first jaw end and a second jaw end, the first jaw end is connected to the first extending portion, and the second jaw end is opposite to the first jaw end;
wherein the lower jaw portion has a third jaw end and a fourth jaw end, the third jaw end is connected to the second extending portion, and the fourth jaw end is opposite to the third jaw end;
wherein a first thickness of the upper jaw portion is gradually decreased from the first jaw end to the second jaw end;
wherein a second thickness of the lower jaw portion is gradually decreased from the third jaw end to the fourth jaw end.

8. A medical anchoring system, comprising:
a medical anchoring device according to claim 1; and
an endoscopic device configured to place the medical anchoring device inside the target cavity and configured to connect the connecting portion of the medical anchoring device to the target object inside the target cavity.

9. The medical anchoring system according to claim 8, wherein the endoscopic device comprises:
a tube portion;
a handle portion connecting the tube portion;
a trigger portion connecting the handle portion; and
a controlling portion disposed at the end of the tube portion;
wherein the medical anchoring device is detachably received at the controlling portion.

10. The medical anchoring system according to claim 8, wherein the connecting portion has a jaw portion configured to connect the target object;
wherein when the trigger portion is triggered, the trigger portion cause the controlling portion to control the jaw portion into an open state.

11. The medical anchoring system according to claim 8, wherein the endoscopic device is configured to make the first puncturing portion puncturing through the first target position of the cavity wall, and the endoscopic device is configured to make the second puncturing portion puncturing through the second target position of the cavity wall.

12. The medical anchoring system according to claim 8, wherein the medical anchoring device is a first medical anchoring device, the connecting portion of the first medical anchoring device is a first connecting portion, the target object is a first target object;
wherein the medical anchoring system comprise a second medical anchoring device, and the second medical anchoring device comprises:
a fifth wire portion having a ninth end and a tenth end;
a sixth wire portion having a eleventh end and a twelfth end;
a fifth puncturing portion connecting to the ninth end of the fifth wire portion and configured to puncture through a fifth target position of the cavity wall of the target cavity;
a sixth puncturing portion connecting to the eleventh end of the sixth wire portion and configured to puncture through a sixth target position of the cavity wall of the target cavity, wherein the sixth target position is different to the fifth target position; and
a second connecting portion connecting to the tenth end of the fifth wire portion and the twelfth end of the sixth wire, the second connecting portion is configured to connect the first target object or a second target object inside the target cavity.

13. The medical anchoring system according to claim 12, wherein the endoscopic device is configured to make the fifth puncturing portion puncturing through the fifth target position of the cavity wall, and the endoscopic device is configured to make the sixth puncturing portion puncturing through the sixth target position of the cavity wall.

14. A method for operating a medical anchoring device, comprising the steps of:
placing the medical anchoring device into a target cavity of a human body by using an endoscopic device, wherein the anchoring device comprises a first wire portion, a second wire portion, a first puncturing portion, a second puncturing portion and a connecting portion, the first puncturing portion connects a first end of the wire portion, the second puncturing portion connects a third second end of the wire portion, and the connecting portion connects a second end of the first wire portion and a fourth end of the second wire portion;
puncturing, by the first puncturing portion, through a first target position of a cavity wall of the target cavity;
puncturing, by the second puncturing portion, through a second target position of the cavity wall of the target cavity, wherein the first target position is different to the second target position;
connecting the connecting portion to a target object inside the target cavity; and
pulling the first wire portion and/or the second wire portion, so as to move the target object to a desired position or move the target object toward a desired direction.

15. The method according to claim 14, wherein the anchoring device comprises a third wire portion and a third puncturing portion, the third puncturing portion is connected to a fifth end of the third wire portion, and a sixth end of the third wire portion is connected to the connecting portion;
wherein the method further comprises the step of:
puncturing, by the third puncturing portion, through a third target position of a cavity wall of the target cavity, wherein the third target position is different to the first target position and the second target position; and
pulling the first wire portion and/or the second wire portion and/or the third puncturing portion, so as to move the target object to the desired position or move the target object toward the desired direction.

16. The method according to claim 14, wherein the anchoring device comprises a fourth wire portion and a fourth puncturing portion, the fourth puncturing portion is connected to a seventh end of the fourth wire portion, and an eighth end of the fourth wire portion is connected to the connecting portion;
wherein the method further comprises the step of:
puncturing, by the fourth puncturing portion, through a fourth target position of a cavity wall of the target cavity, wherein the fourth target position is different to the first target position, the second target position and the third target position; and
pulling the first wire portion and/or the second wire portion and/or the third puncturing portion and/or the fourth puncturing portion, so as to move the target object to the desired position or move the target object toward the desired direction.
